(19) United States
(12) Patent Application Publication
(10) Pub. No.: US 2025/0205420 A1
(43) Pub. Date: Jun. 26, 2025

(54) Subcutaneous Indwelling Needle Assembly

(71) Applicant: AARUY Medical Electronics Co., Ltd., Shenzhen, Guangdong (CN)

(72) Inventor: Qingxu Zhou, Shenzhen, Guangdong (CN)

(21) Appl. No.: 18/852,468

(22) PCT Filed: Jun. 29, 2022
(86) PCT No.: PCT/CN2022/102226

§ 371 Date: Sep. 29, 2024

(30) Foreign Application Priority Data
Apr. 29, 2022 (CN) 202210468463.5

(51) Int. Cl. A61M 5/158

(52) U.S. Cl. CPC A61M 5/158; A61M 2005/1586

(57) ABSTRACT

A subcutaneous indwelling needle assembly, including a base and a needle assembly; the needle assembly includes a puncture needle holder, a puncture needle, an indwelling needle holder, and a soft indwelling needle; the base includes a baseplate for applying on a surface of skin; the baseplate includes a first surface and a second surface provided oppositely; the base is provided with a perforation hole penetrating the first surface and the second surface; on the first surface, an indwelling needle fixing protrusion for insertion of the indwelling needle holder is formed by protruding along a periphery of the perforation hole; and a first restricting part and a second restricting part are provided and cooperatively mounted to restrict a relative rotation of the indwelling needle holder with respect to the indwelling needle fixing protrusion when the indwelling needle holder is inserted into the indwelling needle fixing protrusion.

### FIELD OF THE INVENTION

The present application relates to the technical field of medical products, particularly a subcutaneous indwelling needle assembly.

### BACKGROUND OF THE INVENTION

Subcutaneous indwelling needles may be left under the human skin for a short period of time, eliminating the demand for frequent puncture operations when infusing medication, thus reducing the pain caused by repeated punctures. The commercially available paste-type medicine delivery devices generally involve the use of subcutaneous indwelling needles, such as insulin infusion pumps commonly used by diabetic patients, which continuously infuse insulin subcutaneously to the patient through the insulin pump by simulating the working mechanism of the human pancreas to ensure that the patient has a normal glucose level. Therefore, the use of subcutaneous indwelling needles is particularly important.

Currently, the paste-type medicine delivery device is generally divided into a separate type and an integrated type. The infusion pump of the separate paste-type medicine delivery device is separated from the subcutaneous indwelling needle and connected through the catheter, which requires the pump to be worn on the belt or related accessories, being inconvenient to use and prone to a series of safety problems such as catheter entanglement and clogging. However, the infusion pump of the existing integrated paste-type medicine delivery device, as well as the subcutaneous indwelling needle are provided on one end of the overall structure. As a human body moves, the whole device is driven up and down like a seesaw, and the indwelling needle is just at the front of the seesaw, which is a position with the largest movement travel in the whole device. With irregular movement, it is difficult to avoid enlargement of the patient's skin wound.

### SUMMARY OF THE INVENTION

The objective of the present application is to provide a subcutaneous indwelling needle assembly, so as to improve the reliability of the connection between components in the subcutaneous indwelling needle assembly.

According to an aspect of the present application, provided is a subcutaneous indwelling needle assembly, including a base and a needle assembly; the needle assembly includes a puncture needle holder, a puncture needle, an indwelling needle holder, and a soft indwelling needle; the base includes a baseplate for applying on a surface of skin; the baseplate includes a first surface and a second surface provided oppositely; the base is provided with a perforation hole penetrating the first surface and the second surface; on the first surface, an indwelling needle fixing protrusion for insertion of the indwelling needle holder is formed by protruding along a periphery of the perforation hole; the indwelling needle fixing protrusion is provided with a first restricting part; an exterior wall of the indwelling needle holder is provided with a second restricting part; the first restricting part and the second restricting part, which match each other, are collectively a rotation-stopping mechanism; and the first restricting part and the second restricting part are cooperatively mounted to restrict a relative rotation of the indwelling needle holder with respect to the indwelling needle fixing protrusion when the indwelling needle holder is inserted into the indwelling needle fixing protrusion. When assembling the subcutaneous indwelling needle assembly provided in the present application, the indwelling needle holder is inserted into the indwelling needle fixing protrusion by matching installation between the first restricting part and the second restricting part, which not only ensures a reliable connection between the indwelling needle holder and the base, but also stops a rotation of the indwelling needle holder and the indwelling needle connected to an end of the indwelling needle holder relative to the base. Therefore, after the indwelling needle is inserted into the patient's skin, the indwelling needle is rotated under the patient's skin, which avoids enlargement of the user's skin wound due to irregular movement of the indwelling needle.

Preferably, the first restricting part and the second restricting part are connected by a snap fit.

Preferably, the first restricting part is a first rotation-stopping slot formed by recessing an edge of the indwelling needle fixing protrusion, and the second restricting part is a first rotation-stopping buckle protruding from an exterior wall of the indwelling needle holder.

Preferably, there is a plurality of first restricting parts; the plurality of first restricting parts is evenly distributed at an edge of the indwelling needle fixing protrusion; and the quantity and positional relationship of the second restricting part and the first restricting part correspond to each other. By providing a plurality of evenly distributed first restricting parts and second restricting parts, it is conducive to reinforcing a reliable connection between the indwelling needle holder and the base.

Preferably, an end of the indwelling needle holder proximate to the indwelling needle is provided with a restricting retainment plate; an interior wall of the indwelling needle fixing protrusion is provided with a plurality of elastic restricting retainment buckles; in a direction pointing from the first surface to the second surface, the restricting retainment buckle is tilted in a direction proximate to the indwelling needle; the plurality of restricting retainment buckles are formed as an axial restricting part; a width of an opening of an end of the axial restricting part proximate to the second surface is less than a width of the restricting retainment plate perpendicular to an axis of the indwelling needle; and when the indwelling needle holder is inserted into the indwelling needle fixing platform in a direction pointing from the first surface to the second surface, the restricting retainment plate is protruded from the axial restricting part. In the present solution, when inserting a needle, the indwelling needle holder is inserted into the indwelling needle fixing protrusion in a direction pointing from the first surface to the second surface. The restricting retainment plate of the indwelling needle holder squeezes the restricting retainment buckle so that a free end of the restricting retaining plate moves in a direction distal to the axis of the indwelling needle. The opening of the axial restricting part is thereby stretched open to allow the restricting retainment plate to pass through the axial restricting part. After the restricting retainment plate passes through the axial restricting part, the squeezing effect thereof on the restricting retainment buckle disappears and the restricting retainment buckle is reset, so that the opening of the axial restricting part returns to the size in the natural state. In this case, since a width of the restricting retainment plate is greater than a width of an opening of the axial restricting part, the axial restricting part restricts the restricting retainment plate from passing out of the axial restricting part in a direction pointing from the second surface to the first surface, thereby restricting an axial movement of the indwelling needle holder that is mounted with the base. It may avoid detachment of the indwelling needle holder when the needle is retracted or during use, and it may also restrict movement of the indwelling needle mounted at an end of the indwelling needle holder under the patient's skin.

Preferably, when the indwelling needle holder is inserted into the indwelling needle fixing protrusion, the restricting retainment buckles are abutted against the restricting retainment plate. When the indwelling needle holder is matched and mounted with the base, the indwelling needle holder is clamped into the indwelling needle fixing protrusion of the base by abutting against the restricting retainment plate and the restricting retaining buckle against each other, thereby reinforcing the reliable connection between the indwelling needle holder and the base.

Preferably, an exterior wall of the indwelling needle holder is provided with a third restricting part; the puncture needle holder is provided with a fourth restricting part; the third restricting part and the fourth restricting part, which match each other, are collectively a rotation-stopping mechanism; and the third restricting part and the fourth restricting part are cooperatively mounted to restrict a relative rotation of the indwelling needle holder with respect to the puncture needle holder when the indwelling needle holder is inserted into the puncture needle holder. By matching and mounting the third restricting part and the fourth restricting part, the puncture needle holder stops a rotation relative to the indwelling needle holder, thereby ensuring a reliable connection between the puncture needle holder and the indwelling needle holder, which is conducive to improving the operating speed and the success rate of an operator in inserting an indwelling needle by using a puncture needle connected to one end of the puncture needle holder to pass through the indwelling needle holder.

Preferably, the third restricting part and the fourth restricting part are connected by a snap fit.

Preferably, the third restricting part is a second rotation-stopping buckle protruding from an exterior wall of the indwelling needle holder, and the fourth restricting part is a second rotation-stopping slot formed by recessing an edge of an end of the puncture needle holder proximate to the base.

Preferably, on the second surface, a first annular protrusion is formed around a periphery of the perforation hole, and a second annular protrusion is formed around an exterior of the first annular protrusion. The second surface of the baseplate is a surface of the baseplate for attaching to the patient's skin. On the second surface of the baseplate, the first annular protrusion and the second annular protrusion mentioned above form a double-ring structure around a periphery of the perforation hole. In attaching to the surface of the patient's skin using the second surface with the double-ring structure mentioned above, the double-ring structure creates a suction effect by squeezing the patient's skin so that the skin enters the gap between the double-ring structure, thereby firmly fixing the subcutaneous indwelling needle assembly to the surface of the patient's skin. In addition, it is also capable of providing a good isolation effect between the infusion area and other skin areas, playing a waterproof role.

Preferably, the perforation hole is provided in a middle of the baseplate. Compared to the case in which the perforation hole is provided at an edge of the baseplate, the perforation hole of the present solution is provided in a middle of the baseplate, which enables the movement of the indwelling needle assembly mounted in the perforation hole relative to the skin to be relatively small, and reduces the damage to the skin caused by a large movement of the indwelling needle assembly during use. Additionally, the design satisfies a balanced design for the installation between the indwelling needle assembly and the baseplate, which may improve the reliability of the connection between the indwelling needle assembly and the baseplate.

Preferably, the baseplate is provided with a plurality of insertion device positioning holes penetrating the first surface and the second surface, the plurality of insertion device positioning holes is evenly distributed on a periphery of the perforation hole. An insertion device for use in conjunction with the subcutaneous indwelling needle assembly provided in the present application includes a cover capable of fitting with the positioning holes. The needle assembly is provided within the cover of the insertion device and is capable of being accurately secured to the center of the baseplate by an ejector mechanism within the insertion device when the cover of the insertion device is fitted with the positioning hole on the baseplate.

Preferably, an upper end of the puncture needle holder is provided with an insertion device rotation-stopping slot, which ensures a reliable connection between the puncture needle and the insertion device.

In the subcutaneous indwelling needle assembly provided in the present application, the base for providing the infusion pump and the indwelling needle assembly is integrated into one unit and attached to the user's abdomen, limbs or other locations without additional connection to a tube, which may be directly attached to the skin for use, omitting a long tube, reducing the volume, and increasing the portability, safety and comfort of wearing the device.

### BRIEF DESCRIPTION OF THE DRAWINGS

FIG. 1 is an exploded diagram of components included in the needle assembly;

FIG. 2 is a structural diagram of the first surface of the base;

FIG. 3 is an enlarged diagram of the indwelling-needle protruding platform;

FIG. 4 is a sectional diagram of the subcutaneous indwelling needle assembly in the assembled state;

FIG. 5 is a structural diagram of the second surface of the base.

In the attached drawings mentioned above, the correspondence of each marking is as follows: 1 puncture needle holder; 11 insertion device rotation-stopping slot; 12 second rotation-stopping slot; 2 puncture needle; 3 indwelling needle holder; 31 second rotation-stopping buckle; 32 first rotation-stopping buckle; 33 restricting retainment plate; 4 indwelling needle; 5 protective cover; 6 insertion device positioning hole; 7 baseplate; 71 first surface; 72 second surface; 73 indwelling needle fixing protrusion; 731 first rotation-stopping slot; 732 restricting retainment buckle; 74 first annular protrusion; 75 second annular protrusion.

### DETAILED DESCRIPTION OF THE PREFERRED EMBODIMENTS

The terms “first”, “second”, “third”, “fourth” and the like in the specification, the claims and the above-mentioned drawings of the present application are used to identify different objects and are not intended to describe a particular sequence or order. It should be understood that the ordinal numbers used in such a way are in appropriate cases interchangeable so that the embodiments described herein may be implemented in an order other than that illustrated or described herein. Additionally, the terms “comprise” and “include” and derivatives thereof are intended to be interpreted non-exclusively. For example, a process, method, system, product or apparatus that includes a series of steps or units is not necessarily limited to those that are clearly listed, but may include other steps or units that are not clearly listed or that are inherent to the process, method, product or apparatus.

### Embodiment 1

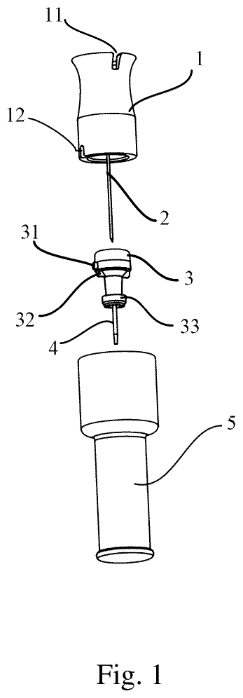

The subcutaneous indwelling needle assembly provided in the present embodiment includes a base and a needle assembly. As shown in FIG. 1, the needle assembly includes a puncture needle holder 1, a puncture needle 2, an indwelling needle holder 3 and a soft indwelling needle 4. An end of the puncture needle holder 1 is fixedly connected to the puncture needle 2, and an end of the indwelling needle holder 3 is fixedly connected to the indwelling needle 4. A bottom exterior wall edge of the puncture needle holder 1 is also provided with a concave second rotation-stopping slot 12. A second rotation-stopping buckle 31 is formed on a top exterior wall of the indwelling needle holder 3 protruding with respect to the exterior wall. A bottom of the puncture needle holder 1 is provided with a groove fitted with a top of the indwelling needle holder 3. When assembling the indwelling needle holder 3 and the puncture needle holder 1, the puncture needle 2 passes through the indwelling needle holder 3 and the indwelling needle 4 sequentially. The second rotation-stopping slot 12 and the second rotation-stopping buckle 31 are snap-fitted with each other, restricting a peripheral rotation of the puncture needle holder 1 with respect to the indwelling needle holder 3. A top of the indwelling needle holder 3 is provided into the groove mentioned above to achieve the abutting of the indwelling needle holder 3 with the puncture needle holder 1, which improves the tightness of the connection between the indwelling needle holder 3 and the puncture needle holder 1, thereby achieving a reliable connection between the puncture needle holder 1 and the indwelling needle holder 3. According to the demand of practical usage, a protective cover 5 may also be provided to match the needle assembly mentioned above. Before assembling the needle assembly and the base, the protective cover 5 is used to sleeve on an exterior of the needle assembly to protect the needle assembly.

In some implementations, an insertion device rotation-stopping slot 11 may also be provided at an upper end of the puncture needle holder 1 for an insertion device matched for use therewith, thereby ensuring a reliable connection between the puncture needle 2 and the insertion device.

In particular, an exterior wall of the indwelling needle holder 3 mentioned above is also provided with a first rotation-stopping buckle 32 protruding with respect to the exterior wall thereof, the first rotation-stopping buckle 32 being provided below the second rotation-stopping buckle 31.

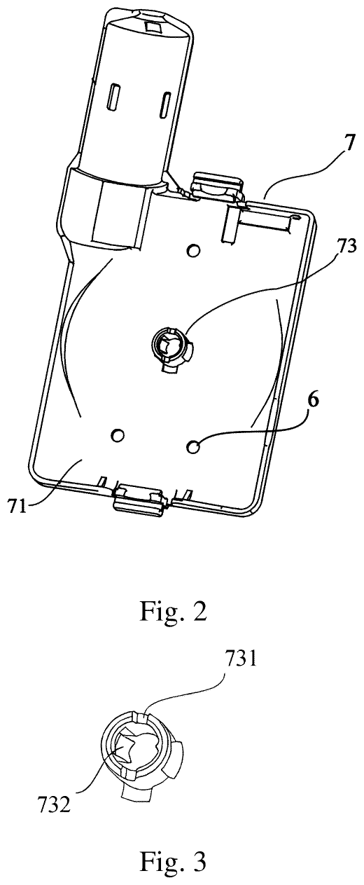

As shown in FIG. 2 and FIG. 3, the base includes a baseplate 7; the baseplate 7 includes a first surface 71 and a second surface 72 provided oppositely; the base is provided with a perforation hole penetrating the first surface 71 and the second surface 72; and on the first surface 71, an indwelling needle fixing protrusion 73 for insertion of the indwelling needle holder 3 is formed by protruding along a periphery of the perforation hole. In the present embodiment, an edge of the indwelling needle fixing protrusion 73 is recessed to form a first rotation-stopping slot 731 that matches the first rotation-stopping buckle 32 mentioned above; the dimensions and the distributing position of the first rotation-stopping slot 731 and the first rotation-stopping buckle 32 match each other. In the present embodiment, the first rotation-stopping slot 731 and the first rotation-stopping buckle 32 are formed in one-to-one correspondence. Two first rotation-stopping slots 731 symmetrically distributed with respect to a central axis of the indwelling needle fixing protrusion 73 are provided on an edge of the indwelling needle fixing protrusion 73. Two first rotation-stopping buckles 32 symmetrically distributed with respect to a central axis of the indwelling needle holder 3 are provided on an exterior wall of the indwelling needle holder 3. When the indwelling needle holder 3 is inserted into the indwelling needle fixing protrusion 73, each of the first rotation-stopping buckles 32 snaps into the first rotation-stopping slot 731 correspondingly provided therewith, thereby restricting the peripheral rotation of the indwelling needle holder 3 with respect to the indwelling needle fixing protrusion 73, and thereby achieving the positioning of the indwelling needle 4 connected to the indwelling needle holder 3 on the base, and avoiding enlargement of the user skin wound area due to irregular movement of the indwelling needle 4 during use.

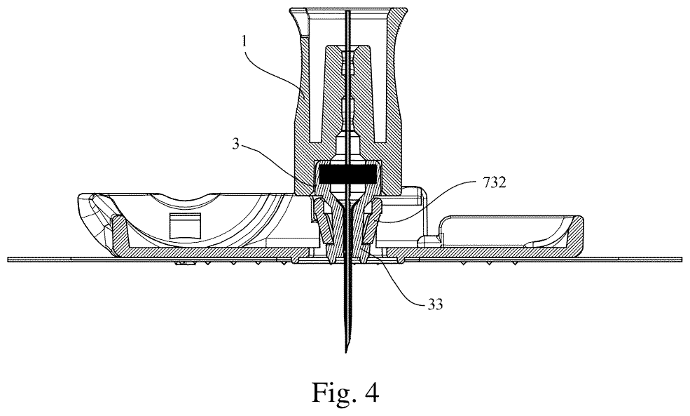

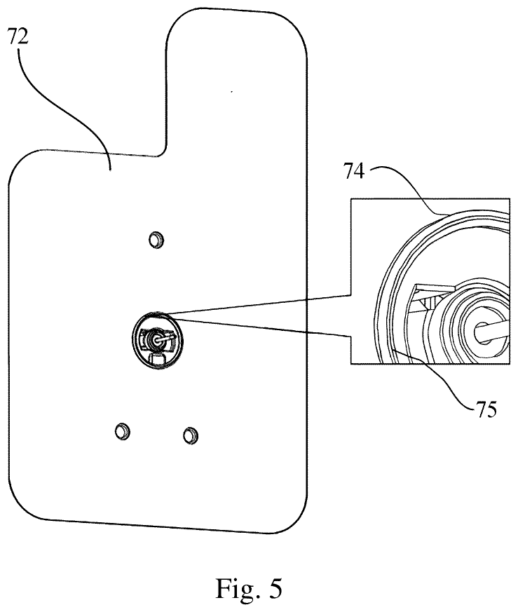

Further, as shown in FIG. 4, an interior wall of the indwelling needle fixing protrusion 73 is also provided with an elastic restricting retainment buckle 732. In a direction pointing from the first surface 71 to the second surface 72, the restricting retainment buckle 732 is tilted in a direction proximal to the indwelling needle 4. In the present embodiment, the amount of the restricting retainment buckles 732 is two; two restricting retainment buckles 732 are symmetrically provided with respect to the center axis of the indwelling needle fixing protrusion 73; and the axial restricting part is formed by two restricting retainment buckles 732 mentioned above. A bottom of the indwelling needle holder 3 is also provided with a restricting retainment plate 33, which in the present embodiment is a rounded platform connected to the indwelling needle 4. A diameter of the restricting retainment plate 33 is greater than the width of an opening at an end of the axial restricting part proximate to the second surface 72. When inserting a needle, the indwelling needle holder 3 is inserted into the indwelling needle fixing protrusion 73 in a direction pointing from the first surface 71 to the second surface 72. The restricting retainment plate 33 of the indwelling needle holder 3 squeezes the restricting retainment buckle 732 so that a free end of the restricting retainment plate 33 moves in a direction distal to an axis of the indwelling needle 4. An opening of the axial restricting part is thereby stretched open to allow the restricting retainment plate 33 to pass through the axial restricting part. After the restricting retainment plate 33 passes through the axial restricting part, the squeezing effect thereof on the restricting retainment buckle 732 is gone and the restricting retainment buckle 732 is reset, so that an opening of the axial restricting part returns to the size in the natural state. In this case, since a width of the restricting retainment plate 33 is greater than a width of an opening of the axial restricting part, the axial restricting part restricts the restricting retainment plate 33 from passing out of the axial restricting part in a direction pointing from the second surface 72 to the first surface 71, thereby restricting an axial movement of the indwelling needle holder 3 that is mounted with the base. It may avoid detachment of the indwelling needle holder 3 when the needle is retracted or during use, and it may also restrict movement of the indwelling needle 4 mounted at an end of the indwelling needle holder 3 under the patient's skin. In the present embodiment, when the indwelling needle holder 3 is inserted into the indwelling needle fixing protrusion 73, the restricting retainment buckles 732 are abutted against the restricting retainment plate 33. Therefore, when the indwelling needle holder 3 is matched and mounted with the base, the indwelling needle holder 3 is clamped into the indwelling needle fixing protrusion 73 of the base by abutting against the restricting baseplate 7 and the restricting retainment buckle 732 against each other, thereby reinforcing the reliable connection between the indwelling needle holder 3 and the base.

Additionally, as shown in FIG. 5, on the second surface 72 of the baseplate 7 adopted by the present embodiment, a first annular protrusion 74 is formed around a periphery of the perforation hole, and a second annular protrusion 75 is formed around an exterior of the first annular protrusion 74. The first annular protrusion 74, the second annular protrusion 75 and the perforation hole are all provided on a same axis. The second surface 72 of the baseplate 7 is a surface of the baseplate 7 for attaching to the patient's skin. On the second surface 72 of the baseplate 7, the first annular protrusion 74 and the second annular protrusion 75 mentioned above form a double-ring structure around a periphery of the perforation hole. In attaching to a surface of the patient's skin using the second surface 72 with the double-ring structure mentioned above, the double-ring structure creates a suction effect by squeezing the patient's skin so that the skin enters a gap between the double-ring structure, thereby firmly fixing the subcutaneous indwelling needle assembly to a surface of the patient's skin. In addition, it is also capable of providing a good isolation effect between the infusion area and other skin areas, playing a waterproof role.

In the present embodiment, the perforation hole is provided in a middle of the baseplate 7. The baseplate 7 is provided with a plurality of insertion device positioning holes 6 penetrating the first surface 71 and the second surface 72, the plurality of insertion device positioning holes 6 are evenly distributed on a periphery of the perforation hole. Compared to the case in which the perforation hole is provided at an edge of the baseplate 7, the perforation hole of the present solution is provided in a middle of the baseplate 7, which enables the movement of the indwelling needle assembly mounted in the perforation hole relative to the skin to be relatively small, and reduces the damage to the skin caused by a large movement of the indwelling needle 4 assembly during use. Additionally, the design satisfies a balanced design for the installation between the indwelling needle 4 assembly and the baseplate 7, which may improve the reliability of the connection between the indwelling needle 4 assembly and the baseplate 7. An insertion device for use in conjunction with the subcutaneous indwelling needle assembly provided in the present application includes a cover capable of fitting with the positioning holes. The needle assembly is provided within the cover of the insertion device and is capable of being accurately secured to a center of the baseplate 7 by an ejector mechanism within the insertion device when the cover of the insertion device is fitted with the insertion device positioning hole 6 on the baseplate 7. Depending on the demand of practical usage, an adhesive tape for attaching to the patient's skin may also be attached to the second surface 72, and the adhesive tape may be attached to the baseplate 7 by ultrasonic welding or adhesive fixation.

The above examples are only used to illustrate the technical solution of the present application rather than to limit the protection scope of the present application. Although the present application has been described in detail with reference to the above examples, a person of ordinary skill in the art should be aware that modifications or equivalent substitutions may be carried out to the technical solution of the present application, these modifications or substitutions fall within the protection scope of the present application.

1. A subcutaneous indwelling needle assembly, comprising a base and a needle assembly;
the needle assembly comprises a puncture needle holder, a puncture needle, an indwelling needle holder, and a soft indwelling needle;
the base comprises a baseplate for applying on a surface of skin; the baseplate comprises a first surface and a second surface provided oppositely; the base is provided with a perforation hole penetrating the first surface and the second surface; on the first surface, an indwelling needle fixing protrusion for insertion of the indwelling needle holder is formed by protruding along a periphery of the perforation hole;
the indwelling needle fixing protrusion is provided with a first restricting part; an exterior wall of the indwelling needle holder is provided with a second restricting part; the first restricting part and the second restricting part, which match each other, are collectively a rotation-stopping mechanism; and the first restricting part and the second restricting part are cooperatively mounted to restrict a relative rotation of the indwelling needle holder with respect to the indwelling needle fixing protrusion when the indwelling needle holder is inserted into the indwelling needle fixing protrusion.

2. The subcutaneous indwelling needle assembly according to claim 1, wherein the first restricting part and the second restricting part are connected by a snap fit.

3. The subcutaneous indwelling needle assembly according to claim 2, wherein the first restricting part is a first rotation-stopping slot formed by recessing an edge of the indwelling needle fixing protrusion, and the second restricting part is a first rotation-stopping buckle protruding from an exterior wall of the indwelling needle holder.

4. The subcutaneous indwelling needle assembly according to claim 3, wherein there is a plurality of first restricting parts; the plurality of first restricting parts is evenly distributed at an edge of the indwelling needle fixing protrusion; and quantity and positional relationship of the second restricting part and the first restricting part correspond to each other.

5. The subcutaneous indwelling needle assembly according to claim 1, wherein an end of the indwelling needle holder proximate to the indwelling needle is provided with a restricting retainment plate; an inner wall of the indwelling needle fixing protrusion is provided with a plurality of elastic restricting retainment buckles; in a direction pointing from the first surface to the second surface, the restricting retainment buckle is tilted in a direction proximate to the indwelling needle;
the plurality of restricting retainment buckles are formed as an axial restricting part; a width of an opening of an end of the axial restricting part proximate to the second surface is less than a width of the restricting retainment plate perpendicular to an axis of the indwelling needle; and when the indwelling needle holder is inserted into the indwelling needle fixing protrusion in a direction pointing from the first surface to the second surface, the restricting retainment plate is protruded from the axial restricting part.

6. The subcutaneous indwelling needle assembly according to claim 5, wherein when the indwelling needle holder is inserted into the indwelling needle fixing protrusion, the restricting retainment buckles are abutted against the restricting retainment plate.

7. The subcutaneous indwelling needle assembly according to claim 1, wherein an exterior wall of the indwelling needle holder is provided with a third restricting part; the puncture needle holder is provided with a fourth restricting part; the third restricting part and the fourth restricting part, which match each other, are collectively a rotation-stopping mechanism; and the third restricting part and the fourth restricting part are cooperatively mounted to restrict a relative rotation of the indwelling needle holder with respect to the puncture needle holder when the indwelling needle holder is inserted into the puncture needle holder.

8. The subcutaneous indwelling needle assembly according to claim 7, wherein the third restricting part and the fourth restricting part are connected by a snap fit.

9. The subcutaneous indwelling needle assembly according to claim 7, wherein the third restricting part is a second rotation-stopping buckle protruding from an exterior wall of the indwelling needle holder, and the fourth restricting part is a second rotation-stopping slot formed by recessing an edge of an end of the puncture needle holder proximate to the base.

10. The subcutaneous indwelling needle assembly according to claim 1, wherein on the second surface, a first annular protrusion is formed around a periphery of the perforation hole, and a second annular protrusion is formed around an exterior of the first annular protrusion.

11. The subcutaneous indwelling needle assembly according to claim 1, wherein the perforation hole is provided in a middle of the baseplate.

12. The subcutaneous indwelling needle assembly according to claim 11, wherein the baseplate is provided with a plurality of insertion device positioning holes penetrating the first surface and the second surface, the plurality of insertion device positioning holes is evenly distributed on a periphery of the perforation hole.
